(19) United States
(12) Patent Application Publication
(10) Pub. No.: US 2021/0165202 A1
(43) Pub. Date: Jun. 3, 2021

(54) EXAMINATION METHOD AND EXAMINATION DEVICE

(71) Applicant: SHIMADZU CORPORATION, Kyoto-shi (JP)

(72) Inventors: Hiroshi IWATA, Kyoto-shi (JP); Shigehiko ZODA, Kyoto-shi (JP); Hiroomi GOTO, Kyoto-shi (JP); Eiichi OZEKI, Kyoto-shi (JP); Yasuko YONEDA, Kyoto-shi (JP); Takahiro NISHIMOTO, Kyoto-shi (JP); Naofumi SAKAUCHI, Kyoto-shi (JP); Ippei TAKEUCHI, Kyoto-shi (JP)

(73) Assignee: SHIMADZU CORPORATION, Kyoto-shi (JP)

(21) Appl. No.: 17/106,340

(22) Filed: Nov. 30, 2020

(30) Foreign Application Priority Data
Nov. 29, 2019 (JP) 2019-216136

(51) Int. Cl. G02B 21/24; H04N 5/232; G02B 21/36; C12Q 1/18

(52) U.S. Cl. CPC G02B 21/244; C12Q 1/18; G02B 21/365; H04N 5/232127

(57) ABSTRACT

One object is to reduce the time required for focusing. When an image capturing condition is not stored (when image capturing of an observation area is performed for the first time), an examination device performs an autofocus process, obtains a focus position where a microscope camera is in focus on the observation area, and stores the obtained focus position in a storage device. In contrast, when the image capturing condition is stored (when image capturing of the observation area is performed for the second and subsequent times), the examination device captures an image of the observation area by reading out the focus position from the storage device and setting a focal point of the microscope camera at the read-out focus position.

### BACKGROUND OF THE INVENTION

### Field of the Invention

The present disclosure relates to an examination method and an examination device.

### Description of the Background Art

Japanese Patent Laying-Open No. 2019-088339 discloses an examination device that performs a bacteria identification examination or a drug susceptibility examination by observing, with a microscope, the shape and the number of bacteria in each well of a culture plate for bacteria identification culture or drug susceptibility examination, and monitoring how the bacteria divide.

### SUMMARY OF THE INVENTION

Due to, for example, the accuracy of molding, there are slight differences among individual plates on which objects to be observed, such as bacteria or drugs, are arranged. Since a depth of field of a microscope is narrow, it is necessary to set a focal point of a microscope camera for each plate.

In monitoring dynamics of a biological factor such as bacteria, images of one observation area are captured a plurality of times. In this case, if a focal point is set every time an image is captured, the time required for one image capturing becomes longer. In addition, when there are a plurality of observation areas and a state of objects to be observed changes over time, it is desirable to capture images of the observation areas included in one plate at the same timing whenever possible. However, when a microscope camera is used, it is necessary to set a focal point for each observation area, and thus, an image capturing timing lag occurs and the lag increases in proportion to an increase in the observation areas. Therefore, the time required to complete image capturing for one plate becomes longer.

The present disclosure has been made to solve the above-described problem, and an object of the present disclosure is to provide an examination method and an examination device that can examine dynamics of a biological factor in a short time.

An examination method of the present disclosure is an examination method for examining dynamics of a biological factor by capturing, with a microscope camera, an image of at least one observation area on a plate with the biological factor arranged therein. The examination method includes: obtaining a focus position by focusing the microscope camera on the observation area during a first image capturing process for the observation area; storing the obtained focus position in a storage device; and performing a second image capturing process for the observation area after the first image capturing process. The performing includes: reading out the focus position from the storage device; setting a focal point of the microscope camera at the read-out focus position; and capturing an image of the observation area.

An examination device of the present disclosure is an examination device that examines dynamics of a biological factor by capturing an image of at least one observation area on a plate with the biological factor arranged therein. The examination device includes: a microscope camera that captures an image of the observation area; a focal point changing unit that changes a focal point of the microscope camera; an obtainment unit that obtains a focus position by focusing the microscope camera on the observation area during a first image capturing process for the observation area; a storage unit that stores the obtained focus position in a storage device; and an image capturing control unit that causes the microscope camera to capture the image of the observation area by controlling the microscope camera and the focal point changing unit. The image capturing control unit performs a second image capturing process for the observation area after the first image capturing process. The second image capturing process includes: reading out the focus position from the storage device; setting the focal point of the microscope camera at the read-out focus position; and capturing an image of the observation area.

The foregoing and other objects, features, aspects and advantages of the present disclosure will become more apparent from the following detailed description of the present disclosure when taken in conjunction with the accompanying drawings.

### BRIEF DESCRIPTION OF THE DRAWINGS

FIG. 1 shows a schematic configuration of an examination device according to the present embodiment.

FIG. 2 is a plan view of a culture plate.

FIG. 3 is a schematic view showing an example hardware configuration of a controller.

FIG. 4 is a block diagram showing an example functional configuration of the controller.

FIG. 5 is a flowchart of an image capturing process performed by the controller.

FIG. 6 is a flowchart of an image capturing process performed by a controller according to a first modification.

FIG. 7 is a flowchart of an image capturing process performed by a controller according to a second modification.

FIG. 8 is a flowchart of a correction process performed by the controller according to the second modification.

### DESCRIPTION OF THE PREFERRED EMBODIMENTS

An embodiment of the present disclosure will be described in detail hereinafter with reference to the drawings, in which the same or corresponding portions are denoted by the same reference characters and description thereof will not be repeated.

[Configuration of Examination Device]

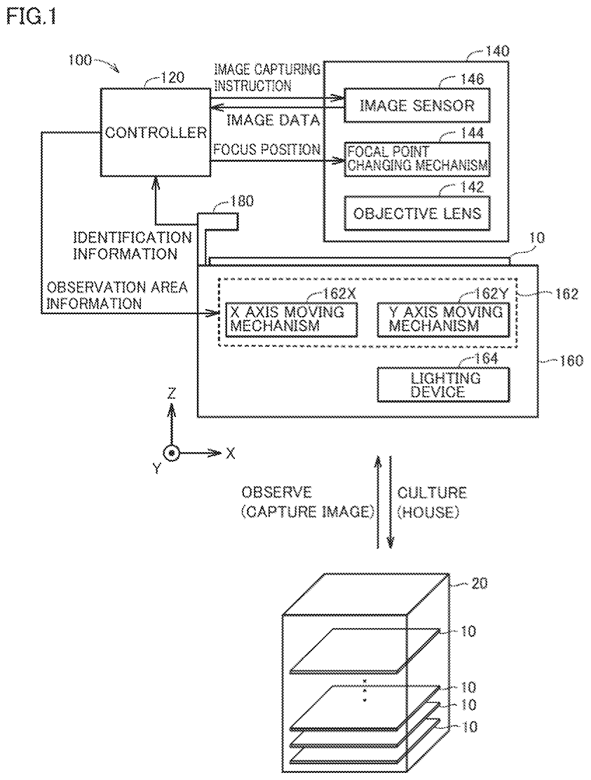

FIG. 1 shows a schematic configuration of an examination device according to the present embodiment. As one example, the examination device according to the present embodiment is used for a drug susceptibility test.

An examination device 100 captures an image of each of a plurality of observation areas provided on a culture plate 10. An object to be observed obtained by bringing a drug into contact with a test solution including bacteria (biological factor) is arranged at each of the plurality of observation areas.

Examination device 100 includes a controller 120, microscope camera 140, a stage 160, and a reading unit 180. Controller 120 is electrically connected to microscope camera 140, stage 160 and reading unit 180. The electrically connected devices may be partly or entirely formed of one piece.

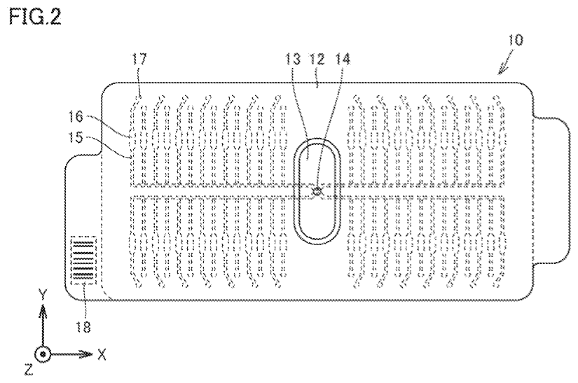

In order to capture an image of each observation area 16 (see FIG. 2) on culture plate 10, controller 120 controls each of microscope camera 140 and stage 160 based on information read by reading unit 180.

Microscope camera 140 includes an objective lens 142, a focal point changing mechanism 144 and an image sensor 146.

Objective lens 142 magnifies a part of culture plate 10 placed on stage 160. Objective lens 142 is arbitrarily selected in accordance with the object to be observed.

Focal point changing mechanism 144 changes a focal point of microscope camera 140. As one example, focal point changing mechanism 144 changes the focal point of microscope camera 140 by changing a position of objective lens 142 in an optical axis direction of objective lens 142.

Image sensor 146 is a detector for capturing an image of the object to be observed magnified by objective lens 142, and is, for example, a charge coupled device (CCD) image sensor, a complementary metal oxide semiconductor (CMOS) image sensor or the like.

Stage 160 includes an image-capturing field-of-view changing mechanism 162 and a lighting device 164. Culture plate 10 is placed on stage 160. Lighting device 164 is transparent lighting and irradiates stage 160 with light for observation.

Image-capturing field-of-view changing mechanism 162 changes an image-capturing field of view of microscope camera 140. Image-capturing field-of-view changing mechanism 162 includes an X axis moving mechanism 162X and a Y axis moving mechanism 162Y. X axis moving mechanism 162X moves culture plate 10 placed on stage 160 in an X axis direction in FIG. 1. Y axis moving mechanism 162Y moves culture plate 10 placed on stage 160 in a Y axis direction in FIG. 1. In FIG. 1, a plane of stage 160 on which culture plate 10 is placed is defined as an X-Y plane, and an axis vertical to the X-Y plane is defined as a Z axis.

Reading unit 180 reads identification information of culture plate 10. Reading unit 180 is, for example, a barcode reader, a QR code (registered trademark) reader or a reader adapted to a radio frequency (RF) tag, and is selected in accordance with the type of an identification code assigned to culture plate 10. Reading unit 180 transmits the read identification information to controller 120.

Controller 120 reads out an image capturing condition corresponding to the identification information based on the identification information from reading unit 180, and controls microscope camera 140 and stage 160 based on the read out image capturing condition, to capture an image of each observation area.

Specifically, controller 120 outputs, to image-capturing field-of-view changing mechanism 162, observation area information indicating a position of the observation area whose image is to be captured. In accordance with the output observation area information, image-capturing field-of-view changing mechanism 162 moves culture plate 10, such that the observation area whose image is to be captured is located within the image-capturing field of view of microscope camera 140.

In addition, controller 120 provides a focal point changing instruction to focal point changing mechanism 144 in accordance with the image capturing condition. At this time, controller 120 outputs, to focal point changing mechanism 144, a focus position where microscope camera 140 is in focus on the observation area whose image is to be captured. Focal point changing mechanism 144 sets the focal point of microscope camera 140 at the output focus position.

Controller 120 provides an image capturing instruction to image sensor 146 when the image-capturing field of view and the focal point of microscope camera 140 are set, and obtains image data. Controller 120 obtains the number of the bacteria, the shape of the bacteria and the like as an observation result from the image data.

[Configuration of Culture Plate]

FIG. 2 is a plan view of the culture plate. Culture plate 10 includes a plate-shaped member 12 and a flow path structure. The flow path structure includes an opening portion 13, an opening 14, a micro flow path 15, observation area 16, and an opening 17.

Opening 14 is a portion provided in opening portion 13 and allowing opening portion 13 and micro flow path 15 to communicate with each other. Namely, opening 14 is connected to one end of micro flow path 15. Using a fluid pressure, the test solution including the bacteria is injected from opening 14 into micro flow path 15. On culture plate 10 shown in FIG. 2, four micro flow paths 15 are arranged radially around opening 14.

Micro flow path 15 is configured such that the test solution can flow therethrough. Micro flow path 15 extending from opening 14 branches off to a plurality of micro flow paths 15. The test solution introduced from opening 14 flows through branched micro flow paths 15. In the present embodiment, one micro flow path 15 branches off to fourteen micro flow paths 15.

Observation area 16 is provided partway through branched micro flow path 15. Micro flow path 15 allows the test solution introduced from opening 14 to flow to observation area 16.

Observation area 16 has the drug arranged thereat, and is connected to micro flow path 15 to store the test solution introduced from micro flow path 15. At observation area 16, the test solution reacts with the drug. The drug is, for example, an antibacterial drug. The drug may be solid, or may be liquid. The drug is preliminarily placed at observation area 16. That is, the drug is placed at observation area 16 before the test solution flows into observation area 16. Observation area 16 is formed to have a rectangular parallelepiped shape. One side of observation area 16 has a length of, for example, 10 μm to 10 mm.

In FIG. 2, fifty-six (=14×4) observation areas 16 are formed on plate-shaped member 12. That is, in the present embodiment, when one culture plate 10 is observed, fifty-six observation areas 16 are observed using an examination device 100. The volumes of the test solutions stored in fifty-six observation areas 16 are the same as each other. In contrast, the types and the amounts of the drugs placed at fifty-six observation areas 16 may be the same as each other, or may be different from each other.

Plate-shaped member 12 is made of an acrylic resin such as a polymethyl methacrylate resin. A thickness of plate-shaped member 12 is not particularly limited, and is set at, for example, 1 mm to 6 mm. In addition, an identification code 18 for individually identifying culture plate 10 is assigned to plate-shaped member 12.

Identification code 18 is not limited to an optically readable code such as a one-dimensional barcode or a two-dimensional QR code (registered trademark), and may be a code that can be read by wireless communication, such as an RF tag. Identification information indicated by identification code 18 is not limited to the serial number individually assigned to culture plate 10, and may be the lot number assigned to culture plate 10.

Culture plate 10 is made mainly of an acrylic resin. Therefore, culture plate 10 has a slight individual difference due to a difference in manufacturing condition, storage condition, use condition, or the like. When a camera having a wide depth of field is used, an image that is in focus to some extent is obtained, regardless of the slight individual difference, by focusing on the same position as a position of a focal point that is focused on when capturing an image of one culture plate 10, and capturing an image of another culture plate 10. However, in the examination according to the present embodiment, microscope camera 140 having a narrow depth of field is used. Therefore, an in-focus image is not obtained by focusing on the same position as a position of a focal point that is focused on when capturing an image of one culture plate 10, and capturing an image of another culture plate 10.

Accordingly, in the examination according to the present embodiment using the microscope camera having a narrow depth of field, information for focusing on each observation area 16 is managed, as an image capturing condition, by the identification information indicated by individual identification code 18 assigned to each culture plate 10.

[Overview of Examination]

An overview of the examination will be described with reference to FIG. 1. In the present embodiment, examination device 100 is used to observe a state of the bacteria after the drug is brought into contact with the bacteria. Culture plate 10 into which the test solution is injected is housed in an incubator 20 set at a temperature (e.g., 37° C.) suitable for culture of the bacteria, and is taken out of incubator 20 at the timing of observation.

Culture plate 10 is housed in incubator 20 for, for example, three hours after the drugs are brought into contact with the bacteria. Then, assuming that the time of bringing the drugs into contact with the bacteria is 0 minute, a state of the bacteria is observed at each of 0 minute, 60 minutes, 90 minutes, 120 minutes, 150 minutes, and 180 minutes. Thus, a change over time in the bacteria when the drugs are brought into contact with the bacteria is observed and a result of examination of dynamics of the bacteria is obtained.

Fifty-six observation areas 16 are provided on culture plate 10. In the examination according to the present embodiment, the process of taking culture plate 10 out of incubator 20, capturing an image of each of fifty-six observation areas 16 on culture plate 10 using examination device 100, and returning culture plate 10 back to incubator 20 is repeated.

A plurality of culture plates 10 may be observed concurrently. In this case, for each culture plate 10, the above-described process of taking culture plate 10 out of incubator 20, capturing an image of each of fifty-six observation areas 16, and returning culture plate 10 back to incubator 20 is repeated.

[Hardware Configuration of Controller]

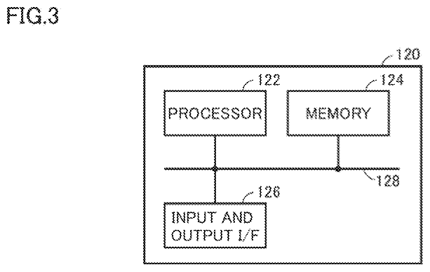

FIG. 3 is a schematic view showing an example hardware configuration of the controller. As one example, controller 120 is formed in accordance with a general-purpose computer architecture.

As main components, controller 120 includes a processor 122, a memory 124, and an input and output interface (I/F) 126. These components are communicably connected to each other through a bus 128.

Processor 122 is typically a processing unit such as a central processing unit (CPU) or a multi processing unit (MPU). Processor 122 reads out and executes a program stored in memory 124, to thereby control the operation of each portion of examination device 100. Specifically, processor 122 executes the program, to thereby implement each process of examination device 100 described below. In the example of FIG. 3, controller 120 includes a single processor. However, controller 120 may include a plurality of processors.

Memory 124 is implemented by a nonvolatile memory such as a random access memory (RAM), a read only memory (ROM) and a flash memory, or a storage device such as a magnetic disk. Memory 124 stores a program executed by processor 122, data used by processor 122, or the like. Specifically, memory 124 stores the image capturing condition for capturing an image of each observation area 16 on each culture plate 10.

Input and output I/F 126 is an interface for exchanging various types of data with focal point changing mechanism 144, image sensor 146, image-capturing field-of-view changing mechanism 162, and reading unit 180.

[Functional Configuration of Controller]

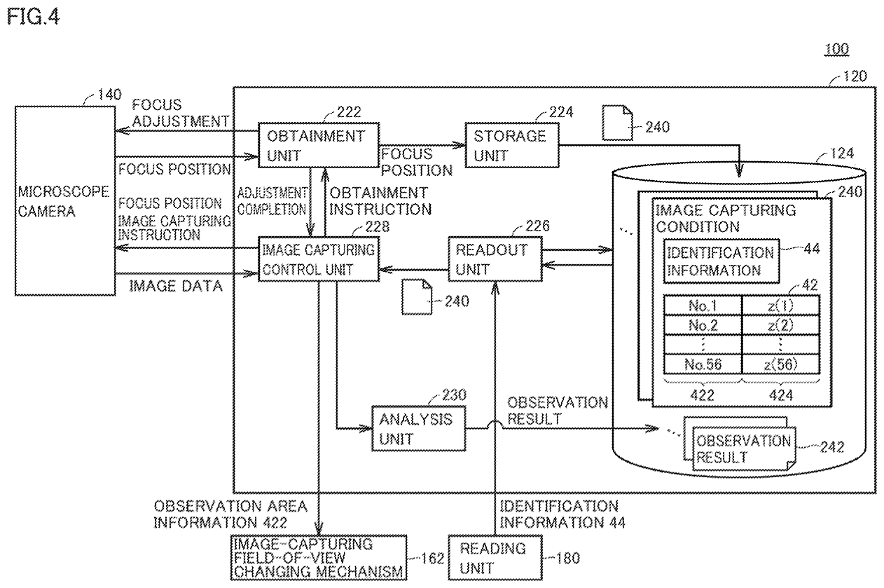

FIG. 4 is a block diagram showing an example functional configuration of the controller. Controller 120 includes an obtainment unit 222, a storage unit 224, a readout unit 226, an image capturing control unit 228, and an analysis unit 230. Each of these functions is implemented by processor 122 executing a program stored in memory 124.

Obtainment unit 222 obtains a focus position 424 where microscope camera 140 is in focus on observation area 16. Using the existing autofocus technique, obtainment unit 222 obtains focus position 424 by focusing microscope camera 140 on observation area 16, in cooperation with focal point changing mechanism 144.

Focus position 424 refers to, for example, a position of objective lens 142 when microscope camera 140 is in focus on observation area 16, and a distance in a Z axis direction from a surface of stage 160 to objective lens 142.

A method for obtaining focus position 424 is not limited to a method using the existing autofocus technique. For example, the focus position may be obtained by measuring a distance from a predetermined position to observation area 16 with a laser-type displacement sensor, calculating a distance from the X-Y plane of stage 160 to observation area 16, and adding a focal length of microscope camera 140 to the calculated distance.

Storage unit 224 stores, as an image capturing condition 240, identification information 44 and camera information 42 in memory 124 in association with each other. Camera information 42 includes observation area information 422 and focus position 424. Storage unit 224 stores, as camera information 42, observation area information 422 and focus position 424 in memory 124 in association with each other, focus position 424 being for focusing microscope camera 140 on observation area 16 indicated by observation area information 422.

Observation area information 422 is information indicating a position of one observation area 16 on culture plate 10. In the present embodiment, observation area information 422 is a number of observation area 16 indicating each of fifty-six observation areas 16 provided on culture plate 10.

Readout unit 226 reads out image capturing condition 240 from memory 124, based on identification information 44 read by reading unit 180. Readout unit 226 transmits the read out result to image capturing control unit 228. When image capturing condition 240 corresponding to identification information 44 is not stored, readout unit 226 outputs, to image capturing control unit 228, a notification that image capturing condition 240 is not stored. When image capturing condition 240 corresponding to identification information 44 is stored, readout unit 226 outputs image capturing condition 240 to image capturing control unit 228.

Image capturing control unit 228 controls microscope camera 140 and image-capturing field-of-view changing mechanism 162, to thereby obtain image data of each observation area 16.

When image capturing control unit 228 receives, from readout unit 226, the notification that image capturing condition 240 is not stored, image capturing control unit 228 obtains the image data while generating image capturing condition 240. Specifically, image capturing control unit 228 outputs, to image-capturing field-of-view changing mechanism 162, the number of observation area 16 that is observation area information 422. Observation area information 422 indicating the position of observation area 16 on culture plate 10 is prestored in memory 124.

In accordance with observation area information 422 output from image capturing control unit 228, image-capturing field-of-view changing mechanism 162 moves culture plate 10, such that observation area 16 having the designated number (position) is located within the image-capturing field of view of microscope camera 140.

In addition, image capturing control unit 228 instructs obtainment unit 222 to obtain focus position 424. In cooperation with focal point changing mechanism 144, obtainment unit 222 obtains focus position 424 by focusing microscope camera 140 on observation area 16 located within the image-capturing field of view of microscope camera 140. At this time, focal point changing mechanism 144 focuses microscope camera 140 on observation area 16 located within the image-capturing field of view of microscope camera 140.

In response to completion of the focus adjustment, image capturing control unit 228 provides an image capturing instruction to image sensor 146 and obtains image data of observation area 16.

When the image capturing is completed, image capturing control unit 228 changes the number of observation area 16 and repeats the instruction to image-capturing field-of-view changing mechanism 162, the instruction to obtainment unit 222, and the image capturing instruction to image sensor 146. By repetition of these instructions by image capturing control unit 228, focus position 424 is obtained, image capturing condition 240 is generated and image data is obtained for each observation area 16.

Analysis unit 230 analyzes the image data obtained by image capturing control unit 228, to thereby obtain an observation result 242. Analysis unit 230 stores obtained observation result 242 in memory 124. Observation result 242 may include, for example, binarized data of the image data, data indicating the number of the bacteria and the shape of the bacteria obtained by analyzing the image data, the image capturing time, observation area information 422 indicating the image capturing position, and the like.

When image capturing condition 240 is output from readout unit 226, image capturing control unit 228 controls microscope camera 140 and image-capturing field-of-view changing mechanism 162 in accordance with output image capturing condition 240 (i.e., read out image capturing condition 240), to thereby obtain image data of each observation area 16. Analysis unit 230 outputs, to memory 124, observation result 242 obtained by analyzing the obtained image data.

Specifically, image capturing control unit 228 outputs the number of the observation area to image-capturing field-of-view changing mechanism 162 and outputs the focus position corresponding to the number of the observation area to focal point changing mechanism 144 of microscope camera 140, and provides the image capturing instruction to image sensor 146 of microscope camera 140.

As a result, observation area 16 having the designated number is located within the image-capturing field of view of microscope camera 140 and microscope camera 140 is in focus on this observation area 16, and thus, the in-focus image data is obtained. At this time, focal point changing mechanism 144 may only set the position of objective lens 142 at the designated focus position and adjustment of the position of objective lens 142 for focusing is unnecessary. Therefore, the time required to focus microscope camera 140 on observation area 16 can be reduced.

As described above, when image capturing of each observation area 16 on culture plate 10 is performed for the first time (a first image capturing process), examination device 100 according to the present embodiment adjusts the position of objective lens 142 to focus microscope camera 140 on observation area 16. When image capturing is performed for the second and subsequent times (a second image capturing process), examination device 100 according to the present embodiment uses the adjustment result obtained in the image capturing performed for the first time. Thus, the time required to perform the image capturing for the second and subsequent times can be reduced. As a result, the time required to obtain the result of the bacteria dynamics examination can be reduced.

Focus position 424 may be obtained for each observation area 16 on culture plate 10 before the test solution is injected into culture plate 10, and then, the test solution may be injected into culture plate 10 and the examination may be started. That is, in the image capturing performed for the first time, examination device 100 may obtain only focus position 424 by focusing microscope camera 140 on observation area 16, without obtaining the observation result. In other words, in the image capturing performed for the first time, examination device 100 may obtain only focus position 424 by focusing microscope camera 140 on observation area 16, without actually performing the image capturing.

As described above, fifty-six observation areas 16 are provided on culture plate 10. Therefore, if the time to complete image capturing of one observation area 16 becomes longer, a time lag between the time when an image of first observation area 16 is captured and the time when an image of last fifty-sixth observation area 16 is captured becomes larger.

In addition, in the case of observing a plurality of culture plates 10 concurrently, if the time to complete image capturing of one observation area 16 becomes longer, the time to complete observation of one culture plate 10 becomes longer. For example, when observation is performed at thirty-minute intervals, it is necessary to finish observation of all culture plates 10 for thirty minutes at the latest. Therefore, if the time to complete observation of one culture plate 10 becomes longer, the number of culture plates 10 that can be processed concurrently becomes smaller.

When one observation area 16 is observed a plurality of times, examination device 100 according to the present embodiment reads identification code 18 assigned to culture plate 10 and sets the focal point of microscope camera 140 by using focus position 424 corresponding to identification information 44 indicated by read identification code 18. Therefore, when one observation area 16 is observed a plurality of times using examination device 100, the time to complete image capturing of one observation area 16 can be reduced, as compared with the case of making an adjustment to focus microscope camera 140 on observation area 16 whenever observation area 16 on culture plate 10 is observed. As a result, the time lag between the time when an image of first observation area 16 is captured and the time when an image of last observation area 16 is captured can be reduced, and the number of culture plates 10 that can be processed concurrently can also be increased.

Although FIG. 4 shows the configuration example in which the necessary functions are provided by processor 122 executing the program, a part or all of these provided functions may be implemented using a dedicated hardware circuit (such as, for example, an application specific integrated circuit (ASIC) or a field-programmable gate array (FPGA)).

[Flowchart]

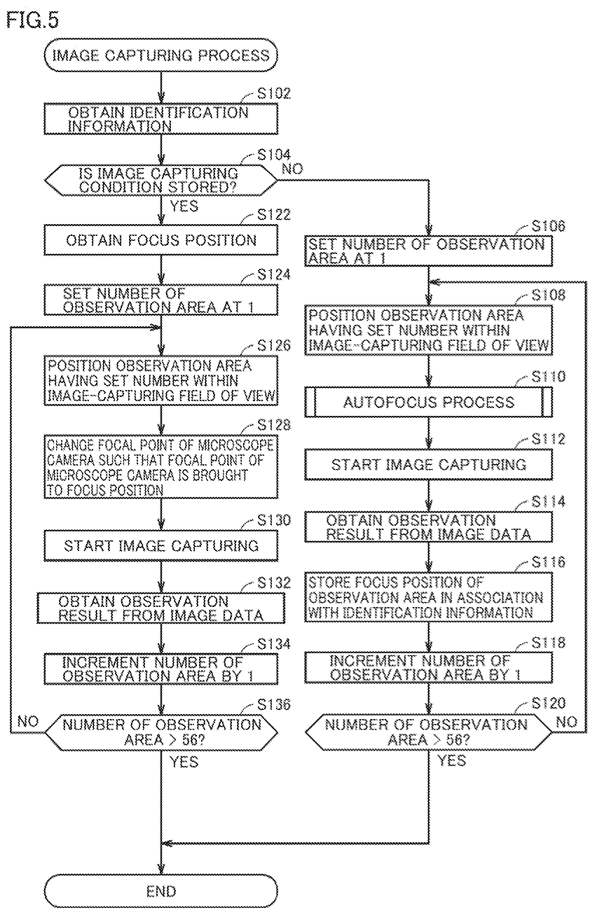

FIG. 5 is a flowchart of an image capturing process performed by the controller. The image capturing process is started when culture plate 10 is placed on stage 160. In the following description, a step will be simply denoted as “S”.

In S102, controller 120 obtains identification information 44. In S104, controller 120 determines whether or not image capturing condition 240 corresponding to obtained identification information 44 is stored in memory 124.

When controller 120 determines that image capturing condition 240 is not stored in memory 124 (NO in S104), controller 120 performs the processing in S106 to S120, and then, ends the image capturing process.

In S106, controller 120 sets the number of observation area 16 at 1. In S108, controller 120 positions observation area 16 having the set number within the image-capturing field of view.

In S110, controller 120 performs an autofocus process. The autofocus process refers to a process of focusing microscope camera 140 on observation area 16 located within the image-capturing field of view of microscope camera 140 using the existing autofocus technique. Focus position 424 is thus obtained.

In S112, controller 120 causes image sensor 146 of microscope camera 140 to start image capturing. In S114, controller 120 obtains an observation result from image data obtained by microscope camera 140. The obtained observation result is stored in memory 124. Controller 120 stores, for example, the image capturing time, the number of observation area 16 set when the image is captured, and information obtained by analyzing the image data in memory 124 as the observation result.

In S116, controller 120 stores focus position 424 obtained by the autofocus process in S110 as focus position 424 of observation area 16 having the set number, in association with identification information 44 obtained by the processing in S102. At this time, controller 120 stores focus position 424 in association with observation area information 422 indicating the set number.

In S118, controller 120 increments the number of observation area 16 by 1. In S120, controller 120 determines whether or not the number of observation area 16 exceeds fifty six. Controller 120 repeats the processing in S108 to S120 until the number of observation area 16 exceeds fifty six, and then, ends the image capturing process.

When controller 120 determines that image capturing condition 240 is stored in memory 124 (YES in S104), controller 120 performs the processing in S122 to S136, and then, ends the image capturing process.

In S122, controller 120 obtains focus position 424 (camera information 42) corresponding to identification information 44 obtained from memory 124 in S102.

In S124, controller 120 sets the number of observation area 16 at 1. In S126, controller 120 positions observation area 16 having the set number within the image-capturing field of view.

In S128, controller 120 changes the focal point of microscope camera 140 such that the focal point of microscope camera 140 is brought to focus position 424 corresponding to the set number (observation area information 422).

In S130, controller 120 causes image sensor 146 of microscope camera 140 to start image capturing. In S132, controller 120 obtains an observation result from image data obtained by microscope camera 140. The obtained observation result is stored in memory 124. Controller 120 stores, for example, the image capturing time, the number of observation area 16 set when the image is captured, and information obtained by analyzing the image data in memory 124 as the observation result.

In S134, controller 120 increments the number of observation area 16 by 1. In S136, controller 120 determines whether or not the number of observation area 16 exceeds fifty six. Controller 120 repeats the processing in S126 to S136 until the number of observation area 16 exceeds fifty six, and then, ends the image capturing process.

The processing in S106 to S120 is the processing performed when the image capturing process is performed on culture plate 10 for the first time (a first image capturing process), and is the processing of concurrently performing generation of image capturing condition 240 and image capturing of each observation area 16. In contrast, the processing in S122 to S136 is the processing performed when the image capturing process has already been performed on culture plate 10, and is the processing performed when image capturing of observation area 16 is performed for the second and subsequent times (a second image capturing process). In the processing in S122 to S136, an image of each observation area 16 is captured using image capturing condition 240 generated by performing the processing in S106 to S120.

[First Modification]

Culture plate 10 is housed in incubator 20, and is taken out at the predetermined timing and placed on stage 160. Since culture plate 10 is made of resin, culture plate 10 is expected to become deformed when culture plate 10 is housed in incubator 20. Accordingly, in image capturing performed for the second and subsequent times, image capturing condition 240 generated in image capturing performed for the first time may be corrected and used. In image capturing performed for the second and subsequent times, controller 120 according to a first modification performs a process of correcting image capturing condition 240 (focus position 424).

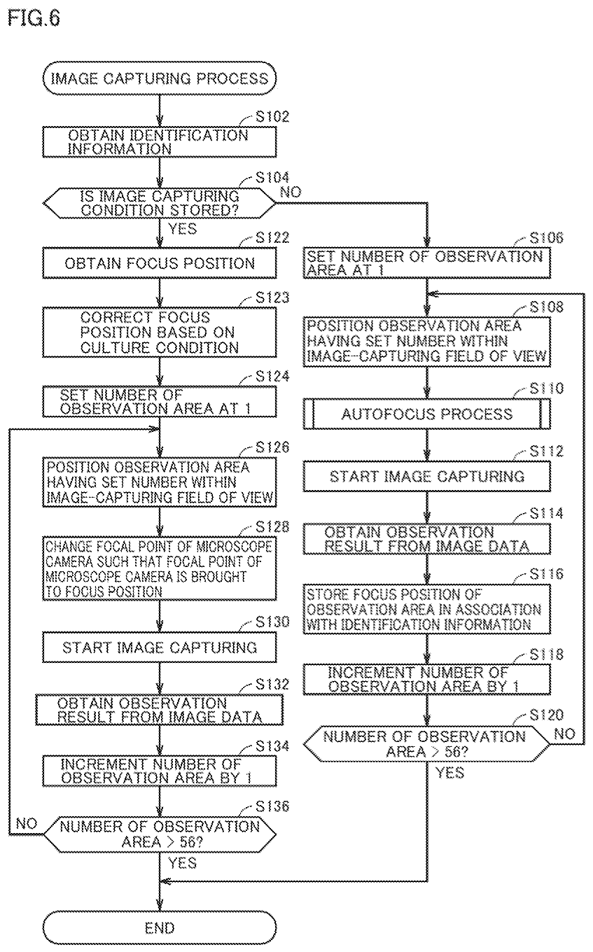

FIG. 6 is a flowchart of an image capturing process performed by a controller according to the first modification. The image capturing process performed by controller 120 according to the first modification is different from the image capturing process performed by controller 120 according to the above-described embodiment in that controller 120 according to the first modification performs the processing in S123.

In S123, controller 120 according to the first modification corrects focus position 424 based on a culture condition. The culture condition is a factor that deforms culture plate 10, and includes a temperature and a humidity of the environment where culture plate 10 is placed, a time period during which culture plate 10 is housed in incubator 20, and the like.

For example, a degree of deformation of culture plate 10 under the environment equivalent to the environment during examination of culture plate 10 may be measured and focus position 424 may be corrected based on the obtained degree of deformation. Alternatively, focus position 424 may be corrected in accordance with a correction equation including the culture condition as a parameter.

The processing in S123 may be performed separately from the image capturing process. The processing in S123 may be performed at any timing after generation of image capturing condition 240.

[Second Modification]

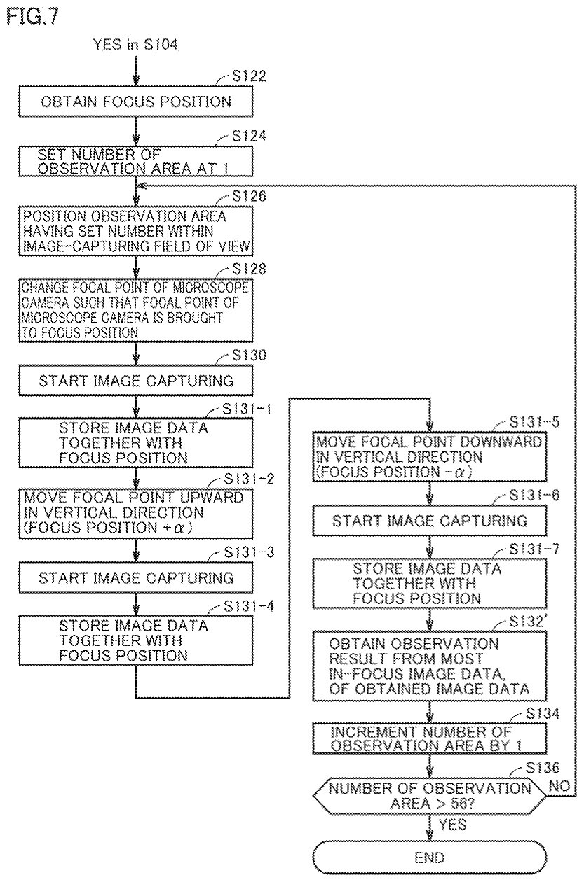

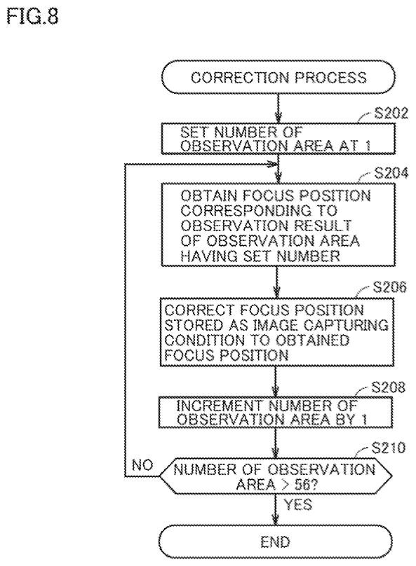

Although controller 120 according to a second modification is common to controller 120 according to the first modification in that controller 120 according to the second modification corrects image capturing condition 240, controller 120 according to the second modification is different in a correction method from controller 120 according to the first modification. A process performed by controller 120 according to the second modification will be described below with reference to FIGS. 7 and 8.

FIG. 7 is a flowchart of an image capturing process performed by the controller according to the second modification. In FIG. 7, a part of the process (processing in S102 to S120) common to the image capturing process performed by the controller according to the above-described embodiment is omitted.

The image capturing process performed by controller 120 according to the second modification is different from the image capturing process performed by controller 120 according to the above-described embodiment in that controller 120 according to the second modification performs the processing in S132′ instead of the processing in S132 and performs the processing in S131-1 to S131-7. The processing different from that of the above-described embodiment will be mainly described below.

Subsequent to S130, in S131-1, controller 120 according to the second modification stores image data together with the focus position.

In S131-2, controller 120 according to the second modification moves the focal point of microscope camera 140 upward in a vertical direction (in the Z axis direction in FIG. 1) by a prescribed distance a. Specifically, the focal point is moved from focus position 424 stored as image capturing condition 240 in a direction away from stage 160 (culture plate 10) by predetermined distance a. The distance of movement may be set in accordance with the depth of field of microscope camera 140, or may be set in accordance with the environment where culture plate 10 is placed.

In S131-3, controller 120 according to the second modification causes image sensor 146 of microscope camera 140 to start image capturing.

In S131-4, controller 120 according to the second modification stores image data together with the focus position. That is, controller 120 according to the second modification stores the image data in association with the position of the focal point of microscope camera 140 when the image data is obtained.

In S131-5, controller 120 according to the second modification moves the focal point of microscope camera 140 downward in the vertical direction (in the Z axis direction in FIG. 1) by prescribed distance a. Specifically, the focal point is moved from focus position 424 stored as image capturing condition 240 in a direction approaching stage 160 (culture plate 10) by predetermined distance a. The distance of movement does not necessarily need to be the same as the distance of movement in S131-2.

In S131-6, controller 120 according to the second modification causes image sensor 146 of microscope camera 140 to start image capturing.

In S131-7, controller 120 according to the second modification stores image data together with the focus position.

In S132′, controller 120 according to the second modification obtains an observation result from the most in-focus image data, of the image data obtained in S131-1, the image data obtained in S131-4 and the image data obtained in S131-7. For example, controller 120 according to the second modification extracts a feature quantity related to focus from the image data. The feature quantity related to focus can be calculated using the existing image processing technique. Controller 120 according to the second modification specifies the most in-focus image data based on the extracted feature quantity. At this time, controller 120 according to the second modification stores the obtained observation result in association with the focus position when the most in-focus image data is obtained.

By performing the processing in S128 to S131-7, the images captured at the positions of the focal point of microscope camera 140 displaced upward and downward in the vertical direction (in the Z axis direction in FIG. 1) with respect to the focus position by the prescribed distance are obtained, in addition to the image captured at the focus position stored as image capturing condition 240.

In addition, by performing the processing in S132′, the most in-focus image is specified, of the image captured at the focus position stored as image capturing condition 240 and the images captured at the positions of the focal point of microscope camera 140 displaced upward and downward in the vertical direction (in the Z axis direction in FIG. 1) with respect to the focus position by the prescribed distance.

The information composed of the image data and the focus position stored in S131-1, S131-4 and S131-7 may be deleted after the most in-focus image data is specified in the processing in S132′.

FIG. 8 is a flowchart of a correction process performed by the controller according to the second modification. In the correction process, image capturing condition 240 is corrected based on the image captured at the focus position and the images captured at the positions displaced upward and downward with respect to the focus position by the prescribed distance.

Specifically, in S202, controller 120 according to the second modification sets the number of observation area 16 at 1.

In S204, controller 120 according to the second modification obtains a focus position corresponding to observation result 242 of observation area 16 having the set number. At this time, by performing the processing in S132′ in FIG. 7 as described above, observation result 242 obtained from the most in-focus image, of the image captured at the focus position stored as image capturing condition 240 and the images captured at the positions of the focal point of microscope camera 140 displaced upward and downward in the vertical direction (in the Z axis direction in FIG. 1) with respect to the focus position by the prescribed distance, is stored in memory 124 in association with the focus position when the most in-focus image is captured.

That is, by performing the processing in S204, there is obtained the focus position indicating the position of the focal point when the most in-focus image is captured, of the image captured at the focus position stored as image capturing condition 240 and the images captured at the positions of the focal point of microscope camera 140 displaced upward and downward in the vertical direction (in the Z axis direction in FIG. 1) with respect to the focus position by the prescribed distance.

In S206, controller 120 according to the second modification corrects the focus position stored as image capturing condition 240 to the focus position obtained in S204.

In S208, controller 120 according to the second modification increments the number of observation area 16 by 1. In S210, controller 120 according to the second modification determines whether or not the number of observation area 16 exceeds fifty six. Controller 120 according to the second modification repeats the processing in S204 to S210 until the number of observation area 16 exceeds fifty six, and then, ends the correction process.

When culture plate 10 is deformed under the culture environment, the images captured at the positions of the focal point of microscope camera 140 displaced upward and downward along the vertical direction of microscope camera 140 by the prescribed distance may in some cases be more in-focus than the image captured at the focus position stored as image capturing condition 240.

Accordingly, by performing the correction process shown in FIG. 8, the focus position stored as image capturing condition 240 can be corrected to the focus position indicating the position of the focal point of microscope camera 140 when the in-focus image is captured.

The correction process may be performed at any timing during a time period from the n-th image capturing to the n+1-th image capturing. Alternatively, the correction process may be performed when a preset condition is satisfied, such as when a time period during which culture plate 10 is housed in incubator 20 becomes equal to or longer than a prescribed time period. The condition for performing the correction process is set based on, for example, a condition that causes deformation of culture plate 10.

The correction process may be performed whenever image capturing is performed the prescribed number of times. Culture plate 10 is gradually deformed with the progress of the culture time. Therefore, the position of the focal point of microscope camera 140 where microscope camera 140 is in focus on observation area 16 changes gradually with the progress of the culture time. By performing the correction process whenever image capturing is performed the prescribed number of times, image capturing condition 240 can be corrected in accordance with the position of the focal point that changes gradually with the progress of the culture time.

Although controller 120 according to the second modification performs image capturing at three different focal point positions in the image capturing process, the number of the focal point positions may be two or more and is not limited to three.

Controller 120 according to the second modification may perform the processing in S132′ at the timing different from the timing of the image capturing process. For example, the processing in S132′ may be incorporated into a part of the correction process shown in FIG. 8. The correction process may be performed during the image capturing process. Specifically, the processing in S206 may be performed after the processing in S132′.

[Other Modifications]

Although controller 120 stores image capturing condition 240 in memory 124 of controller 120, a location to store image capturing condition 240 is not limited thereto. For example, image capturing condition 240 may be stored in the storage device communicably connected to controller 120. When identification code 18 is a writable RF tag, image capturing condition 240 may be stored in the RF tag.

Although the plurality of observation areas 16 are provided on culture plate 10 in the above-described embodiment, the present disclosure is not limited thereto. The number of observation areas 16 provided on culture plate 10 may be one, or may be two or more, or may be fifty seven or more. When one observation area 16 is provided on culture plate 10, image capturing condition 240 does not need to include the information indicating the position of observation area 16, and image-capturing field-of-view changing mechanism 162 does not need to be provided.

Although the information indicating the position of observation area 16 is the number of observation area 16 in the above-described embodiment, the present disclosure is not limited thereto. For example, the information indicating the position of observation area 16 may be a coordinate on the plane (X-Y plane) of stage 160 on which culture plate 10 is placed. That is, the information indicating the position of observation area 16 may be any information indicating a relative positional relationship between microscope camera 140 and observation area 16.

Although observation area information 422 is prestored in memory 124, observation area information 422 may be read when identification code 18 is read.

Although image-capturing field-of-view changing mechanism 162 changes the image-capturing field of view of microscope camera 140 by moving stage 160 in the above-described embodiment, the present disclosure is not limited thereto. For example, image-capturing field-of-view changing mechanism 162 may change the image-capturing field of view of microscope camera 140 by moving microscope camera 140.

In the above-described embodiment, description has been given of the example in which the result obtained by analysis of the image data by analysis unit 230 is stored as observation result 242. However, the present disclosure is not limited thereto. Controller 120 does not need to include analysis unit 230 and may store the image data as observation result 242. Alternatively, controller 120 may transmit the image data to another information processing device and cause the information processing device to have the function of the analysis unit. Although observation result 242 is stored in memory 124 in the above-described embodiment, observation result 242 may be represented on a display unit such as a monitor and observation result 242 may be deleted in response to an input indicating that a user has checked the representation.

Although microscope camera 140 includes focal point changing mechanism 144 in the above-described embodiment, the present disclosure is not limited thereto. For example, focal point changing mechanism 144 may be any mechanism as long as it can change the position of the focal point of microscope camera 140 with respect to culture plate 10, and may be, for example, a mechanism that moves stage 160 in the Z axis direction.

In the above-described embodiment, the image capturing condition for focusing on each observation area 16 is managed by the identification information indicated by individual identification code 18 assigned to each culture plate 10. However, a method for managing the image capturing condition for each culture plate 10 is not limited to the method using the identification information. For example, the image capturing condition for each culture plate 10 may be managed using information of a position (storage position) where each culture plate 10 is stored. In this case, it is unnecessary to assign identification code 18 to culture plate 10, and it is also unnecessary to provide reading unit 180 in examination device 100.

For example, readout unit 226 may read out the image capturing condition corresponding to the storage position, based on reception of an input of the storage position. In this case, the image capturing condition may be deleted at the timing of performing a new examination or at the timing of the end of examination. The storage position refers to, for example, a position in the incubator. The storage position may be output by the user operating a prescribed input unit. Alternatively, when the process of taking culture plate 10 in and out of the incubator is performed by a machine, the storage position may be output from a controller that controls the machine. A placement position during observation of culture plate 10, not the storage position, may be predetermined for each culture plate 10 and the image capturing condition for each culture plate 10 may be managed using the placement position.

When one culture plate 10 is examined at a time, it is unnecessary to manage the image capturing condition using the identification information.

[Aspects]

It is understood by a person skilled in the art that the above-described embodiment and the modifications thereof are provided as specific examples of the following aspects.

(Clause 1)

An examination method according to one aspect is an examination method for examining dynamics of a biological factor by capturing, with a microscope camera, an image of at least one observation area on a plate with the biological factor arranged therein. The examination method includes: obtaining a focus position by focusing the microscope camera on the observation area during a first image capturing process for the observation area; storing the obtained focus position in a storage device; and performing a second image capturing process for the observation area after the first image capturing process. The performing includes: reading out the focus position from the storage device; setting a focal point of the microscope camera at the read-out focus position; and capturing an image of the observation area.

With such a configuration, in the second image capturing process for the observation area after the first image capturing process, the focal point of the microscope camera is set at the focus position obtained in the first image capturing process. That is, it is unnecessary to focus the microscope camera on the observation area in the second image capturing process for the observation area. As a result, the time required for focusing can be reduced and the dynamics examination of the biological factor can be performed in a short time.

(Clause 2)

The examination method according to clause 1 further includes reading identification information assigned to the plate. In this case, the storing the obtained focus position further includes storing the focus position in association with the identification information. The reading out further includes reading out the focus position corresponding to the read identification information.

With such a configuration, each focus position is stored in the storage device in association with the identification information assigned to the plate, and thus, the focus position where the microscope camera is in focus on the observation area can be managed for each plate having an individual difference. Thus, the examination can be simplified when a plurality of plates are observed concurrently.

(Clause 3)

In the examination method according to clause 1 or 2, the plate may have a plurality of observation areas. In this case, the storing further includes storing observation area information in association with the focus position, the observation area information indicating a relative positional relationship between the microscope camera and the observation area. The performing further includes setting an image-capturing field of view of the microscope camera in accordance with the observation area information corresponding to the read out-focus position.

With such a configuration, the time required for focusing for each observation area can be reduced. Therefore, the plurality of observation areas can be observed concurrently and an image capturing timing lag among the observation areas can be reduced.

(Clause 4)

In the examination method according to any one of clauses 1 to 3, the biological factor is bacteria. In this case, an object to be observed is arranged at the observation area, the object to be observed being obtained by bringing an antibacterial drug into contact with a sample including the bacteria.

(Clause 5)

The examination method according to clause 4 may further include: housing the plate in an incubator for a predetermined time period; and correcting the focus position stored in the storage device in accordance with a culture condition.

With such a configuration, although the plate is expected to become deformed when the plate is housed in the incubator, the focus position is corrected in accordance with the culture condition, and thus, obtainment of the image that is in focus on the observation area can be continued.

(Clause 6)

The examination method according to clause 4 may further include: housing the plate in an incubator for a predetermined time period; capturing an image of the observation area at a position of the focal point of the microscope camera displaced from the focus position in a vertical direction with respect to the plate by a prescribed distance; and obtaining an observation result using an in-focus image, of the image captured at the focus position and the image captured at the position displaced in the vertical direction by the prescribed distance.

With such a configuration, although the plate is expected to become deformed when the plate is housed in the incubator, image capturing at the focus position and image capturing at the position displaced in the vertical direction by the prescribed distance are performed, and thus, the observation result using the image that is in focus on the observation area is obtained even when the plate is deformed.

(Clause 7)

The examination method according to clause 4 may further include: housing the plate in an incubator for a predetermined time period; capturing an image of the observation area at a position of the focal point of the microscope camera displaced from the focus position in a vertical direction with respect to the plate by a prescribed distance; and correcting the focus position stored in the storage device to a position of the focal point of the microscope camera in capturing an in-focus image, of the image captured at the focus position and the image captured at the position displaced in the vertical direction by the prescribed distance.

With such a configuration, although the plate is expected to become deformed when the plate is housed in the incubator, the focus position is corrected to the position of the focal point of the microscope camera in capturing the in-focus image, of the image captured at the focus position and the image captured at the position displaced in the vertical direction by the prescribed distance, and thus, obtainment of the in-focus image can be continued.

(Clause 8)

An examination device according to one aspect is an examination device that examines dynamics of a biological factor by capturing an image of at least one observation area on a plate with the biological factor arranged therein. The examination device includes: a microscope camera that captures an image of the observation area; a focal point changing unit that changes a focal point of the microscope camera; an obtainment unit that obtains a focus position by focusing the microscope camera on the observation area during a first image capturing process for the observation area; a storage unit that stores the obtained focus position in a storage device; and an image capturing control unit that causes the microscope camera to capture the image of the observation area by controlling the microscope camera and the focal point changing unit. The image capturing control unit performs a second image capturing process for the observation area after the first image capturing process. The second image capturing process includes: reading out the focus position from the storage device; setting the focal point of the microscope camera at the read-out focus position; and capturing an image of the observation area.

With such a configuration, in the second image capturing process for the observation area after the first image capturing process, the focal point of the microscope camera is set at the focus position obtained in the first image capturing process. That is, it is unnecessary to focus the microscope camera on the observation area in the second image capturing process for the observation area. As a result, the time required for focusing can be reduced and the dynamics examination of the biological factor can be performed in a short time.

(Clause 9)

The examination device according to clause 8 may further include a reading unit that reads identification information assigned to the plate. In this case, the storage unit stores the focus position in the storage device in association with the identification information. The image capturing control unit reads out the focus position corresponding to the identification information read by the reading unit from the storage device in the second image capturing.

With such a configuration, each focus position is stored in the storage device in association with the identification information assigned to the plate, and thus, the focus position where the microscope camera is in focus on the observation area can be managed for each plate having an individual difference. Thus, the examination can be simplified when a plurality of plates are observed concurrently.

(Clause 10)

The examination device according to clause 8 or 9 may further include an image-capturing field-of-view changing unit that changes an image-capturing field of view of the microscope camera. The plate may have a plurality of observation areas. In this case, the storage unit stores observation area information in association with the focus position, the observation area information indicating a relative positional relationship between the microscope camera and the observation area. The image capturing control unit causes the image-capturing field-of-view changing unit to set an image-capturing field of view of the microscope camera by controlling the image-capturing field-of-view changing unit in accordance with the observation area information corresponding to the read-out focus position.

With such a configuration, the time required for focusing for each observation area can be reduced. Therefore, the plurality of observation areas can be observed concurrently and an image capturing timing lag among the observation areas can be reduced.

(Clause 11)

In the examination device according to any one of clauses 8 to 10, when the image of the observation area on the plate taken out of an incubator is captured, the image capturing control unit may correct the focus position stored in the storage device in accordance with a culture condition of an object to be observed arranged on the observation area.

With such a configuration, although the plate is expected to become deformed when the plate is housed in the incubator, the focus position is corrected in accordance with the culture condition, and thus, obtainment of the image that is in focus on the observation area can be continued.

(Clause 12)

In the examination device according to any one of clauses 8 to 10, the image capturing control unit may obtain a first image and a second image when the image of the observation area on the plate taken out of an incubator is captured, the first image being an image captured at the focus position, the second image being an image captured at a position displaced from the focus position in a vertical direction with respect to the plate by a prescribed distance, and obtain an observation result using an in-focus image, of the first image and the second image.

With such a configuration, although the plate is expected to become deformed when the plate is housed in the incubator, image capturing at the focus position and image capturing at the position displaced in the vertical direction by the prescribed distance are performed, and thus, the observation result using the image that is in focus on the observation area is obtained even when the plate is deformed.

(Clause 13)

In the examination device according to any one of clauses 8 to 10, the image capturing control unit may obtain a first image and a second image when the image of the observation area on the plate taken out of an incubator is captured, the first image being an image captured at the focus position, the second image being an image captured at a position displaced from the focus position in a vertical direction with respect to the plate by a prescribed distance, and correct the focus position stored in the storage device to a position of the focal point of the microscope camera in capturing an in-focus image, of the first image and the second image.

With such a configuration, although the plate is expected to become deformed when the plate is housed in the incubator, the focus position is corrected to the position of the focal point of the microscope camera at the time of capturing the in-focus image, of the image captured at the focus position and the image captured at the position displaced in the vertical direction by the prescribed distance, and thus, obtainment of the in-focus image can be continued.

Although the embodiment of the present disclosure has been described, it should be understood that the embodiment disclosed herein is illustrative and non-restrictive in every respect. The scope of the present disclosure is defined by the terms of the claims and is intended to include any modifications within the scope and meaning equivalent to the terms of the claims.

What is claimed is:

1. An examination method for examining dynamics of a biological factor by capturing, with a microscope camera, an image of at least one observation area on a plate with the biological factor arranged therein, the examination method comprising:
obtaining a focus position by focusing the microscope camera on the observation area during a first image capturing process for the observation area;
storing the obtained focus position in a storage device; and
performing a second image capturing process for the observation area after the first image capturing process,
the performing including:
reading out the focus position from the storage device;
setting a focal point of the microscope camera at the read-out focus position; and
capturing an image of the observation area.

2. The examination method according to claim 1, further comprising reading identification information assigned to the plate, wherein
the storing the obtained focus position further including storing the focus position in association with the identification information, and
the reading out further including reading out the focus position corresponding to the read identification information.

3. The examination method according to claim 1, wherein
the plate has a plurality of observation areas,
the storing further including storing observation area information in association with the focus position, the observation area information indicating a relative positional relationship between the microscope camera and the observation area, and
the performing further including setting an image-capturing field of view of the microscope camera in accordance with the observation area information corresponding to the read-out focus position.

4. The examination method according to claim 1, wherein
the biological factor is bacteria, and
an object to be observed is arranged at the observation area, the object to be observed being obtained by bringing an antibacterial drug into contact with a sample including the bacteria.

5. The examination method according to claim 4, further comprising:
housing the plate in an incubator for a predetermined time period; and
correcting the focus position stored in the storage device in accordance with a culture condition.

6. The examination method according to claim 4, further comprising:
housing the plate in an incubator for a predetermined time period;
capturing an image of the observation area at a position of the focal point of the microscope camera displaced from the focus position in a vertical direction with respect to the plate by a prescribed distance; and
obtaining an observation result using an in-focus image, of the image captured at the focus position and the image captured at the position displaced in the vertical direction by the prescribed distance.

7. The examination method according to claim 4, further comprising:
housing the plate in an incubator for a predetermined time period;
capturing an image of the observation area at a position of the focal point of the microscope camera displaced from the focus position in a vertical direction with respect to the plate by a prescribed distance; and
correcting the focus position stored in the storage device to a position of the focal point of the microscope camera in capturing an in-focus image, of the image captured at the focus position and the image captured at the position displaced in the vertical direction by the prescribed distance.

8. An examination device that examines dynamics of a biological factor by capturing an image of at least one observation area on a plate with the biological factor arranged therein, the examination device comprising:
a microscope camera that captures an image of the observation area;
a focal point changing unit that changes a focal point of the microscope camera;
an obtainment unit that obtains a focus position by focusing the microscope camera on the observation area during a first image capturing process for the observation area;
a storage unit that stores the obtained focus position in a storage device; and
an image capturing control unit that causes the microscope camera to capture the image of the observation area by controlling the microscope camera and the focal point changing unit, wherein
the image capturing control unit performs a second image capturing process for the observation area after the first image capturing process;
the second image capturing process includes:
reading out the focus position from the storage device;
setting the focal point of the microscope camera at the read-out focus position; and
capturing an image of the observation area.

9. The examination device according to claim 8, further comprising
a reading unit that reads identification information assigned to the plate, wherein
the storage unit stores the focus position in the storage device in association with the identification information, and
the image capturing control unit reads out the focus position corresponding to the identification information read by the reading unit from the storage device in the second image capturing.

10. The examination device according to claim 8, further comprising
an image-capturing field-of-view changing unit that changes an image-capturing field of view of the microscope camera, wherein
the plate has a plurality of observation areas,
the storage unit stores observation area information in association with the focus position, the observation area information indicating a relative positional relationship between the microscope camera and the observation area, and
the image capturing control unit causes the image-capturing field-of-view changing unit to set the image-capturing field of view of the microscope camera by controlling the image-capturing field-of-view changing unit in accordance with the observation area information corresponding to the read-out focus position.

11. The examination device according to claim 8, wherein
when the image of the observation area on the plate taken out of an incubator is captured, the image capturing control unit corrects the focus position stored in the storage device in accordance with a culture condition of an object to be observed arranged on the observation area.

12. The examination device according to claim 8, wherein
the image capturing control unit
obtains a first image and a second image when the image of the observation area on the plate taken out of an incubator is captured, the first image being an image captured at the focus position, the second image being an image captured at a position displaced from the focus position in a vertical direction with respect to the plate by a prescribed distance, and
obtains an observation result using an in-focus image, of the first image and the second image.

13. The examination device according to claim 8, wherein
the image capturing control unit
obtains a first image and a second image when the image of the observation area on the plate taken out of an incubator is captured, the first image being an image captured at the focus position, the second image being an image captured at a position displaced from the focus position in a vertical direction with respect to the plate by a prescribed distance, and
corrects the focus position stored in the storage device to a position of the focal point of the microscope camera in capturing an in-focus image, of the first image and the second image.
